(19) United States
(12) Patent Application Publication
(10) Pub. No.: US 2025/0359889 A1
(43) Pub. Date: Nov. 27, 2025

(54) METHOD AND DEVICE FOR TREATING THE SKIN

(71) Applicant: Venus Concept Inc., Toronto (CA)

(72) Inventors: Almog Zahavi, Tel Aviv (IL); Yoni Iger, Haifa (IL); Anat Kotler, Herzliya (IL); Genadi Denenburg, Holon (IL)

(73) Assignee: Venus Concept Inc., Toronto, ON (CA)

(21) Appl. No.: 18/872,580

(22) PCT Filed: Jun. 6, 2023
(86) PCT No.: PCT/IB2023/055838

§ 371 Date: Dec. 6, 2024

Related U.S. Application Data
(60) Provisional application No. 63/349,602, filed on Jun. 7, 2022.
(60) Provisional application No. 63/349,604, filed on Jun. 7, 2022.
(60) Provisional application No. 63/349,600, filed on Jun. 7, 2022.
(60) Provisional application No. 63/349,601, filed on Jun. 7, 2022.

(51) Int. Cl. A61B 17/3205; A61B 17/00; A61B 18/00; A61B 18/14; A61N 2/00

(52) U.S. Cl. CPC A61B 17/32053; A61B 18/1402; A61N 2/002; A61N 2/004; A61B 2017/00026; A61B 2017/00061; A61B 2017/00084; A61B 2017/00212; A61B 2017/00561; A61B 2017/00761; A61B 2017/00893; A61B 2018/0047; A61B 2018/00577; A61B 2018/00589; A61B 2018/00791; A61B 2018/00875

(57) ABSTRACT

A method for treating a patient's skin is provided. The method includes identifying at least one region of said patient's skin in need of treatment. The method includes forming at least one interference of at least one tissue portion in said region of skin, thereby generating at least one scar-like fibrous tissue in said region of said patient's skin. Rotational Fractional Resection (“RFR”) is a procedure which may be used to achieve focal aesthetic contouring by removing fractions of lax skin and excess fat tissue from a patient. Skin may be removed by the use of a rotating micro-coring punch, which is a hollow, sharpened tube which excises full thickness dermal resections. Such punch has been adapted to treat, among other conditions, scars, acne scars, lines, wrinkles, stretch marks, melasma, and to improve skin texture and tighten the skin.

### CROSS-REFERENCE TO RELATED APPLICATION

The present application claims priority to U.S. Provisional Application No. 63/349,601 filed Jun. 7, 2022, the entire contents of which are incorporated herein by reference.

### BACKGROUND OF THE INVENTION

This invention relates to methods and devices for skin treatment. More specifically, this invention relates to methods and devices for skin coring and tightening that would benefit from endorsing collagen synthesis, formation of collagen growth and providing directional skin tightening in said skin tissue thus, providing skin restoration or tightening.

Excess tissue and skin laxity are of wide concern in aesthetic medicine.

Accordingly, there is still a long felt need for effective and efficient approaches to treating the skin that will provide long lasting results with minimized trauma. These approaches should be associated with predictable results and be relatively easy to employ.

### SUMMARY OF THE INVENTION

In aesthetic medicine, elimination of excess tissue and/or skin laxity is an important concern that affects more than 25% of the U.S. population. Conventional surgical therapies (e.g., a face lift, brow lift, or breast lift) can be effective but are often invasive, inconvenient, and expensive, while scarring limits their applicability.

Removing 5%- 15% of skin in an area through excising a multitude of <1 mm diameter cores of dermis and applying directional compression elastic bandages has been shown to provide skin tightening that can be tuned in a desired direction without (noticeable) scarring. An automated robotic dermal micro-coring system with machine vision and robotic precision delivers accuracy, repeatability, and efficiency that provides high value to medical clinics.

Methods using energy sources (e.g., laser, non-coherent light, radiofrequency, or ultrasound) can be effective at improving the architecture and the texture of the skin but are much less effective at tightening the skin or reducing skin laxity. Neurotoxins, such as botulinum toxin, reduce the formation of dynamic wrinkles by paralysis of the injected muscles, but such toxins have minimal or no effect on skin tightness or laxity. Finally, dermal fillers, such as hyaluronic acid, are provided (e.g., injected) in the dermal layer to smooth out wrinkles and improve contours, but such fillers do not tighten or reduce laxity of the skin. Thus, surgical therapies remain the gold standard for lifting and/or tightening skin.

Rotational Fractional Resection (“RFR”) is a procedure which may be used to achieve focal aesthetic contouring by removing fractions of lax skin and excess fat tissue from a patient. Skin may be removed by the use of a rotating micro-coring punch, which is a hollow, sharpened tube which excises full thickness dermal resections. Such punch has been adapted to treat, among other conditions, scars, acne scars, lines, wrinkles, stretch marks, melasma, and to improve skin texture and tighten the skin. As the punch create tiny diameter punctures in the skin; such puncture triggers the body's wound healing process; thereby give the treated area healing process with less discoloration and/or deformation and greater smoothness of the surface.

However, such methods are not problem-free and there is still a need to enhance efficacy thereof. Thus, there is a need for improved methods and devices that increase the effectiveness of such minimally-invasive techniques. Furthermore, there is still a long felt need for an automated including robotic system for dermal micro-coring to be used in minimally invasive directional skin tightening procedures.

It is one object of the present invention to provide a method for treating a patient's skin, comprising steps of:
i. identifying at least one region of said patient's skin in need with treatment; and,
ii. forming at least one interference of at least one tissue portion in said region of skin thereby generating at least one scar-like fibrous tissue in said region of said patient's skin;
wherein said step of generating at least one scar-like fibrous tissue in said region of said patient's skin treats said at least one region.

It is another object of the present invention to provide the method as defined above, wherein said step of forming at least one interference of at least one tissue portion comprising generating at least one scar tissue in at least one tissue portion in said region of skin.

It is another object of the present invention to provide the method as defined above, wherein said step of forming at least one interference of at least one tissue portion comprising at least one step selected from a group consisting of step of excising at least one tissue portion; step of coring at least one tissue portion; step of incision of at least one tissue portion and any combination thereof.

It is another object of the present invention to provide the method as defined above, wherein said step of forming at least one interference of at least one tissue portion results in generating a plurality of scar-like fibrous tissue in said region of skin, each at a different angle, relative to each other and said skin.

It is another object of the present invention to provide the method as defined above, wherein said step of forming at least one interference of at least one tissue portion is performed at an angle A with respect to the surface of said region of skin.

It is another object of the present invention to provide the method as defined above, wherein said angle A is in the range of about 0 to about 90 degrees.

It is another object of the present invention to provide the method as defined above, wherein said step of forming at least one interference of at least one tissue portion results in the generation of crisscross structure in said at least one tissue portion.

It is another object of the present invention to provide the method as defined above, wherein said step of identifying at least one region of said patient's skin with said at least one region additionally comprising step of scanning said region of said patient's skin.

It is another object of the present invention to provide the method as defined above, additionally comprising step of storing said scanned data; and, analyzing thereof so that the efficacy of a treatment can be assessed.

It is another object of the present invention to provide the method as defined above, additionally comprising step of providing recommendations as to where, on said region of skin, to produce said step of forming at least one interference of at least one tissue portion, based on efficacy of a treatments of a plurality of patients.

It is another object of the present invention to provide the method as defined above, further comprising step of confirming that the generated scar-like fibrous tissue is associated with the said treatment.

It is another object of the present invention to provide the method as defined above, additionally comprising step of forming at least one interference of at least one tissue portion in said region of skin.

It is another object of the present invention to provide the method as defined above, additionally comprising step of applying contraction or expansion tension to said region of skin tissue before and/or after said step of forming at least one interference of at least one tissue portion.

It is another object of the present invention to provide the method as defined above, wherein said application of contraction or expansion tension to said region of skin tissue is provided by at least one selected from a group consisting of Tegaderm®, pressure bandages and any combination thereof.

It is another object of the present invention to provide the method as defined above, wherein said step of applying tension therebetween said two portions applies force in the range of 0 N/mm²-50 N/mm².

It is another object of the present invention to provide the method as defined above, wherein said tension applied in said step of applying tension therebetween said two portions is adjustable based on at least one parameter selected from a group consisting of skin type, age of the patient, type of treatment, anatomy, lesion condition, treated anatomy and any combination thereof.

It is another object of the present invention to provide the method as defined above, wherein said step of applying tension therebetween said two portions is performed at a direction selected from a group consisting of x-, y-, and/or z-direction and any combination thereof.

It is another object of the present invention to provide the method as defined above, wherein said step of forming at least one interference of at least one tissue portion in said region of skin is performed by means selected from a group consisting of mechanical means, application of temperature to heat and evacuate tissue, application of temperature to heat, application of heat to accelerate collagen synthesis in the tissue, application of laser, insertion of threads, pulsed electromagnetic field, RF, coblation, coagulation, ablation, microwave energy, ultrasound, any destruction or disruption to the tissue, application of any other type of energy, any additives to the tissue and any combination thereof.

It is another object of the present invention to provide the method as defined above, wherein said step of forming at least one interference of at least one tissue portion in said region of skin is performed by a system comprising at least one robotic arm, said at least one robotic arm comprising at least one skin interference instrument.

It is another object of the present invention to provide the method as defined above, wherein said at least one skin interference instrument comprising at least one selected from a group consisting of at least one needle, at least one punch, at least one thread, and any combination thereof; said at least one skin interference instrument is configured to contact a surface of the skin to generate at least one interference in the skin tissue.

It is another object of the present invention to provide the method as defined above, wherein at least a portion of said at least one skin interference instrument is disposable.

It is another object of the present invention to provide the method as defined above, wherein at least two skin interference instruments are adapted to penetrate said skin either in a simultaneously or sequentially manner.

It is another object of the present invention to provide the method as defined above, wherein at least two skin interference instruments are characterized by either a similar or substantially different cross section area.

It is another object of the present invention to provide the method as defined above, wherein said at least one skin interference instrument is adapted to penetrate said skin to a depth of 1 to 20 mm.

It is another object of the present invention to provide the method as defined above, wherein said at least one skin interference instrument is characterized by a diameter of 0.15 mm-2.0 mm.

It is another object of the present invention to provide the method as defined above, wherein said cross section area is selected from a group consisting of circular, rectangular, triangular, hexagonal, oval, staggered rows, parallel rows, a spiral pattern, a square or rectangular pattern, a radial distribution and any combination thereof.

It is another object of the present invention to provide the method as defined above, wherein said system additionally comprising at least one controller adapted to control the positioning and orientation of said at least one robotic arm relatively to said skin area.

It is another object of the present invention to provide the method as defined above, wherein said controller comprising at least one engine adapted to control at least one parameter selected from a group consisting of the rotation, translation, angle of said at least one robotic arm relatively to said skin, exact location of impact, depth of penetration, coverage rate, the diameter of at least one excised tissue multiplied by number of cores, different area of said skin to be treated and any combination thereof.

It is another object of the present invention to provide the method as defined above, wherein said parameters are adjusted manually by the operator or automatically by said controller.

It is another object of the present invention to provide the method as defined above, wherein said parameters are real time adjusted.

It is another object of the present invention to provide the method as defined above, wherein said rotation is at a speed in the range of 1000-10000 RPM.

It is another object of the present invention to provide the method as defined above, wherein said translation is at a speed in the range of 0-3000 mm/sec.

It is another object of the present invention to provide the method as defined above, wherein said translation of said at least one robotic arm relatively to said skin changes as said at least one robotic arm gets closer to said skin.

It is another object of the present invention to provide the method as defined above, wherein said rotation of said at least one robotic arm changes as said at least one robotic arm gets closer to said skin and penetrates said skin.

It is another object of the present invention to provide the method as defined above, wherein each one skin interference instrument rotates individually in a predefined direction in a predetermined speed.

It is another object of the present invention to provide the method as defined above, wherein at least two of said at least one skin interference instrument rotate simultaneously.

It is another object of the present invention to provide the method as defined above, wherein each one skin interference instrument translates individually.

It is another object of the present invention to provide the method as defined above, wherein at least two of said skin interference instruments translate simultaneously.

It is another object of the present invention to provide the method as defined above, wherein the distance between each pair of neighboring skin interference instruments is configured to vary and be adjustable either before or during treatment.

It is another object of the present invention to provide the method as defined above, wherein said controller comprises a stopper adapted to limit the depth to which at least a portion of said skin interference instrument penetrates said skin.

It is another object of the present invention to provide the method as defined above, additionally comprising step of sensing contact with said skin.

It is another object of the present invention to provide the method as defined above, wherein said angle of said at least one robotic arm is in the range of about 0 to about 90 degrees.

It is another object of the present invention to provide the method as defined above, wherein said controller is adapted to define at least one no-fly zone; said no-fly zone being defined as an area to which said system provides no treatment.

It is another object of the present invention to provide the method as defined above, further comprising at least one force sensor to determine when said at least one skin interference element penetrates the skin.

It is another object of the present invention to provide the method as defined above, additionally comprising step of delivering additives to the skin.

It is another object of the present invention to provide the method as defined above, wherein said additives are selected from a group consisting of threads, therapeutic agents, anesthesia, saline solution growth factors, platelet-derived growth factor (PDGF), transforming growth factor beta (TGF-β), fibroblast growth factor (FGF), epidermal growth factor (EGF), and keratinocyte growth factor); one or more stem cells; steroids, agents which prevent post-inflammatory skin hyperpigmentation, hydroquinone, azelaic acid, kojic acid, mandelic acid, or niacinamide; one or more analgesics; one or more antifungals; one or more anti-inflammatory agents, or a mineralocorticoid agent, an immune selective anti-inflammatory derivative; one or more antimicrobials; a foam; or a hydrogel, one or more antiseptics, one or more antiproliferative agents, one or more emollients; one or more hemostatic agents, a procoagulant, an anti-fibrinolytic agent, one or more procoagulative, one or more anticoagulative agents, one or more immune modulators, including corticosteroids and non-steroidal immune modulators, one or more proteins; or one or more vitamins, hyaluronic acid, collagen, low melting agarose (LMA), polylactic acid (PLA), and/or hyaluronic acid, hyaluranon); a photosensitizer (e.g., Rose Bengal, riboflavin-5-phosphate (R-5-P), methylene blue (MB), N-hydroxypyridine-2-(1H)-thione (N-HTP), a porphyrin, or a chlorin, as well as precursors thereof); a photochemical agent, 1,8 naphthalimide); a synthetic glue (e.g., a cyanoacrylate adhesive, a polyethylene glycol adhesive, or a gelatin-resorcinol-formaldehyde adhesive), a biologic sealant and any combination thereof.

It is another object of the present invention to provide the method as defined above, wherein said system additionally comprising at least one imaging subsystem adapted to guide said at least one skin interference instrument.

It is another object of the present invention to provide the method as defined above, wherein said imaging subsystem comprises at least one selected from a group consisting at least one camera, under-skin imaging such as ultrasound-based imaging, OCT and any combination thereof.

It is another object of the present invention to provide the method as defined above, wherein said system additionally comprising at least one subsystem selected from a group consisting of (a) vacuum subsystem adapted to apply suction to remove excising portions of said skin tissue; (b) at least one retainer, in communication with at least one excisor configured to produce a plurality of excised tissue portions, adapted to contain said excised tissue, to avoid the use of vacuum; (c) any combination thereof.

It is another object of the present invention to provide the method as defined above, wherein said skin is part of a treatment area selected from a group consisting of buttocks, lower limbs, abdomen, forehead, cheeks, jaw line,, upper arms, tummy, abdomen, face, eyelid, nose, forehead, chin, forehead, lips, nose, neck, thighs, chest, legs, back and any combination thereof.

It is another object of the present invention to provide the method as defined above, wherein said system utilizes at least one selected from a group consisting of mechanical visualization, OCT, Ultrasound, machine learning algorithms, artificial intelligence, image processing and any combination thereof to efficiency select the preferred location of the tissue to be treated to enhance outcome of said treatment.

It is another object of the present invention to provide the method as defined above, additionally comprising step of stabilizing said at least one scar-like fibrous tissue generated.

It is another object of the present invention to provide the method as defined above, wherein said step of stabilizing is performed by application of at least one selected from a group consisting of RF energy, pulsed electromagnetic field, microwave, optical energy, ultrasound, and any combination thereof.

It is another object of the present invention to provide the method as defined above, additionally comprising step of application of at least one thread after and/or before and/or during said step of forming at least one interference of at least one tissue portion in said region of skin; thereby stabilizing said at least one scar-like fibrous tissue generated.

It is another object of the present invention to provide the method as defined above, additionally comprising step of providing at least one cutting element adapted to grind said excised tissue so as to facilitate extraction thereof.

It is another object of the present invention to provide the method as defined above, additionally comprising step of communicating said at least one skin interference instrument with at least one RF generator, adapted to apply RF energy to the skin and tissue, so as to fractional ablate/coagulate the tissue.

It is another object of the present invention to provide the method as defined above, wherein said application of RF energy is either simultaneously or sequentially with said at least one skin interference instrument.

It is another object of the present invention to provide the method as defined above, additionally comprising step of providing at least one dynamic magnetic field pulses to said skin.

It is another object of the present invention to provide the method as defined above, wherein said electromagnetic pulses and said RF energy are provided simultaneously to said skin.

It is another object of the present invention to provide the method as defined above, wherein said RF energy results in heating said skin.

It is another object of the present invention to provide the method as defined above, additionally comprising step of application of at least one energy selected from a group consisting of laser, pulsed electromagnetic field, RF, coblation, coagulation, ablation, microwave energy, ultrasound, application of any other type of energy and any combination thereof is either simultaneously or sequentially applied with said at least one skin interference instrument.

It is another object of the present invention to provide the method as defined above, wherein at least one of the following is being held true (a) the shape of said electromagnetic pulse is selected from the group consisting of square wave, a sine wave, a triangular wave, sawtooth wave, ramp waves, spiked wave or any combination thereof; (b) the magnetic field intensity B of each pulse applied by said pulsed electromagnetic frequency generator ranges between about 0 and about 3 Tesla; (c) the magnetic field intensity B of each pulse applied by said pulsed electromagnetic frequency generator ranges between about 0 to 40 Gauss; (d) the duration of each pulse applied by said pulsed electromagnetic frequency generator ranges between about 3 and about 1000 milliseconds; (e) the frequency F applied by the pulses of said pulsed electromagnetic frequency generator ranges between about 1 Hz and about 40 MHz; (f) the energy E applied by the pulses of said pulsed electromagnetic frequency generator ranges between about 1 and about 150 watts per pulse or any combination thereof; (g) the frequency F applied by the pulses applied by said step of applying pulsed electromagnetic therapy to said region to be higher than about 1 and lower than about 1M Hz; (h) the frequency F applied by said electromagnetic field pulses ranges between 1 Hz and 50 Hz; (i) the frequency of said RF energy ranges between 200 kHz and 10 MHz; (j) the power P applied by said RF energy pulses ranges between 1 W and 100 W of RMS average power; and any combination thereof.

It is another object of the present invention to provide the method as defined above, additionally comprising step of cooling, said skin.

It is another object of the present invention to provide the method as defined above, additionally comprising step of providing at least one treatment substance to the treatment area.

It is another object of the present invention to provide the method as defined above, additionally comprising step of at least partially severing at least one scar-like fibrous tissue.

It is another object of the present invention to provide the method as defined above, wherein said step of at least partially severing at least one scar-like fibrous tissue is performed by said step of forming at least one interference of at least one tissue portion in said region of skin.

It is another object of the present invention to provide a system of treating a patient's skin, comprising:
(i) means for identifying at least one region of said patient's skin in need with treatment; and,
(ii) means for forming at least one interference of at least one tissue portion of skin thereby generating at least one scar-like fibrous tissue in said region of said patient's skin;
wherein said at least one scar-like fibrous tissue generated in said region of said patient's skin treats said at least one region.

It is another object of the present invention to provide the system as defined above, wherein said means of forming at least one interference of at least one tissue portion comprising means for generating at least one scar tissue in at least one tissue portion in said region of skin.

It is another object of the present invention to provide the system as defined above, wherein said means of forming at least one interference of at least one tissue portion comprising at least one selected from a group consisting of means of excising at least one tissue portion; means of coring at least one tissue portion; means of incision of at least one tissue portion and any combination thereof.

It is another object of the present invention to provide the system as defined above, wherein said means of forming at least one interference of at least one tissue portion results in generating a plurality of scar-like fibrous tissue in said region of skin, each at a different angle, relative to each other and said skin.

It is another object of the present invention to provide the system as defined above, wherein said means of forming at least one interference of at least one tissue portion at least a portion of said tissue portion is performed at an angle A with respect to the surface of said region of skin.

It is another object of the present invention to provide the system as defined above, wherein said angle A is in the range of about 0 to about 90 degrees.

It is another object of the present invention to provide the system as defined above, wherein said means of forming at least one interference of at least one tissue portion results in the generation of crisscross structure in said at least one tissue portion.

It is another object of the present invention to provide the system as defined above, wherein said means of identifying at least one region of said patient's skin in need with treatment additionally comprising means of scanning said region of said patient's skin.

It is another object of the present invention to provide the system as defined above, additionally comprising means of storing said scanned data; and, analyzing thereof so that the efficacy of a treatment can be assessed.

It is another object of the present invention to provide the system as defined above, additionally comprising means of providing recommendations as to where, on said region of skin, based on efficacy of a treatments of a plurality of patients.

It is another object of the present invention to provide the system as defined above, further comprising means of confirming that the generated scar-like fibrous tissue is associated with said treatment.

It is another object of the present invention to provide the system as defined above, additionally comprising means of applying contraction or expansion tension to said region of skin tissue before and/or after said step of forming at least one interference of at least one tissue portion.

It is another object of the present invention to provide the system as defined above, wherein said application of contraction or expansion tension to said region of skin tissue is provided by at least one selected from a group consisting of Tegaderm®, pressure bandages and any combination thereof.

It is another object of the present invention to provide the system as defined above, wherein said directional skin tightening is performed at a direction selected from a group consisting of the x-, y-, and/or z-direction and any combination thereof.

It is another object of the present invention to provide the system as defined above, wherein said means of forming at least one interference of at least one tissue portion in said region of skin comprising means selected from a group consisting of mechanical means, application of temperature to heat and evacuate tissue, application of temperature to heat, application of laser, RF, insertion of threads, pulsed electromagnetic field, application of heat to accelerate collagen synthesis in the tissue, coblation, ablation, coagulation, microwave energy, ultrasound, any destruction or disruption to the tissue, application of any other type of energy and any other type of energy, any additives to the tissue and any combination thereof.

It is another object of the present invention to provide the system as defined above, wherein said means of forming at least one interference of at least one tissue portion in said region of skin comprising a system comprising at least one robotic arm, said at least one robotic arm comprising at least one skin interference instrument.

It is another object of the present invention to provide the system as defined above, wherein said at least one skin interference instrument comprising a plurality of punches configured to contact a surface of the skin to generate holes in the skin tissue.

It is another object of the present invention to provide the system as defined above, wherein said at least one skin interference instrument are disposable.

It is another object of the present invention to provide the system as defined above, wherein said plurality of said at least one skin interference instrument are adapted to penetrate said skin either in a simultaneously or sequentially manner.

It is another object of the present invention to provide the system as defined above, wherein said plurality of said at least one skin interference instrument are characterized by either a similar or substantially different cross section area.

It is another object of the present invention to provide the system as defined above, wherein said plurality of said at least one skin interference instrument are adapted to penetrate said skin to a depth of 1 to 20 mm.

It is another object of the present invention to provide the system as defined above, wherein at least a portion of said plurality of said at least one skin interference instruments are characterized by a diameter of 0.15 mm-2.0 mm.

It is another object of the present invention to provide the system as defined above, wherein said cross section area is selected from a group consisting of circular, rectangular, triangular, hexagonal, oval, staggered rows, parallel rows, a spiral pattern, a square or rectangular pattern, a radial distribution and any combination thereof.

It is another object of the present invention to provide the system as defined above, wherein said system additionally comprising at least one controller adapted to control the positioning and orientation of said at least one robotic arm relatively to said skin area.

It is another object of the present invention to provide the system as defined above, wherein said controller comprising at least one engine adapted to control at least one parameter selected from a group consisting of the rotation, translation, angle of said at least one robotic arm relatively to said skin, exact location of impact, depth of penetration, coverage rate, the diameter of at least one excised tissue multiplied by number of cores, different area of said skin to be treated and any combination thereof.

It is another object of the present invention to provide the system as defined above, wherein said parameters are adjusted manually by the operator or automatically by said controller.

It is another object of the present invention to provide the system as defined above, wherein said parameters are real time adjusted.

It is another object of the present invention to provide the system as defined above, wherein said rotation is at a speed in the range of 1000-10000 RPM.

It is another object of the present invention to provide the system as defined above, wherein said translation is at a speed in the range of 0-3000 mm/sec.

It is another object of the present invention to provide the system as defined above, wherein said translation of said at least one robotic arm relatively to said skin changes as said at least one robotic arm gets closer to said skin.

It is another object of the present invention to provide the system as defined above, wherein said rotation of said at least one robotic arm changes as said at least one robotic arm gets closer to said skin and penetrates said skin.

It is another object of the present invention to provide the system as defined above, wherein each punch of said plurality of said at least one skin interference instrument rotates individually in a predefined direction in a predetermined speed.

It is another object of the present invention to provide the system as defined above, wherein said plurality of said at least one skin interference instrument rotate simultaneously.

It is another object of the present invention to provide the system as defined above, wherein each punch of said plurality of said at least one skin interference instrument translates individually.

It is another object of the present invention to provide the system as defined above, wherein said plurality of said at least one skin interference instrument translate simultaneously.

It is another object of the present invention to provide the system as defined above, wherein the distance between each of said at least one skin interference instrument can vary and be adjustable either before or during treatment.

It is another object of the present invention to provide the system as defined above, wherein said controller comprising stopping mechanism adapted to limit the depth to which at least a portion of said plurality of said at least one skin interference instrument penetrate said skin.

It is another object of the present invention to provide the system as defined above, additionally comprising at least one sensor adapted to indicate contact with said skin.

It is another object of the present invention to provide the system as defined above, wherein said angle of said at least one robotic arm to said skin is in the range of about 0 to about 90 degrees.

It is another object of the present invention to provide the system as defined above, wherein said controller is adapted to define at least one no-fly zone; said no-fly zone is defined as an area to which said system provides no treatment.

It is another object of the present invention to provide the system as defined above, further comprising at least one force sensor to determine when said at least one skin interference element penetrates into the skin.

It is another object of the present invention to provide the system as defined above, wherein said system additionally provide the skin with additives.

It is another object of the present invention to provide the system as defined above, wherein said additives are selected from a group consisting of threads, therapeutic agents, anesthesia, saline solution growth factors, platelet-derived growth factor (PDGF), transforming growth factor beta (TGF-β), fibroblast growth factor (FGF), epidermal growth factor (EGF), and keratinocyte growth factor); one or more stem cells; steroids, agents which prevent post-inflammatory skin hyperpigmentation, hydroquinone, azelaic acid, kojic acid, mandelic acid, or niacinamide; one or more analgesics; one or more antifungals; one or more anti-inflammatory agents, or a mineralocorticoid agent, an immune selective anti-inflammatory derivative; one or more antimicrobials; a foam; or a hydrogel, one or more antiseptics, one or more antiproliferative agents, one or more emollients; one or more hemostatic agents, a procoagulant, an anti-fibrinolytic agent, one or more procoagulative, one or more anticoagulative agents, one or more immune modulators, including corticosteroids and non-steroidal immune modulators, one or more proteins; or one or more vitamins, hyaluronic acid, collagen, low melting agarose (LMA), polylactic acid (PLA), and/or hyaluronic acid, hyaluranon); a photosensitizer (e.g., Rose Bengal, riboflavin-5-phosphate (R-5-P), methylene blue (MB), N-hydroxypyridine-2-(1H)-thione (N-HTP), a porphyrin, or a chlorin, as well as precursors thereof); a photochemical agent, 1,8 naphthalimide); a synthetic glue (e.g., a cyanoacrylate adhesive, a polyethylene glycol adhesive, or a gelatin-resorcinol-formaldehyde adhesive), a biologic sealant and any combination thereof.

It is another object of the present invention to provide the system as defined above, wherein said system additionally comprising at least one imaging subsystem adapted to guide said at least one skin interference instrument.

It is another object of the present invention to provide the system as defined above, wherein said imaging subsystem comprises at least one selected from a group consisting at least one camera, under skin imaging such as ultrasound-based imaging, OCT and any combination thereof.

It is another object of the present invention to provide the system as defined above, wherein said system additionally comprising at least one subsystem selected from a group consisting of (a) vacuum subsystem adapted to apply suction to remove excising portions of said skin tissue; (b) at least one retention element, in communication with at least one of said means for producing a plurality of excised tissue portions, adapted to contain said excised tissue, to avoid the use of vacuum; (c) any combination thereof.

It is another object of the present invention to provide the system as defined above, wherein said skin could be part of a treatment area selected from a group consisting of buttocks, lower limbs, abdomen, forehead, cheeks, jaw line, neck, upper arms, tummy, abdomen, face, eyelid, nose, forehead, chin, forehead, lips, nose, neck, thighs, chest, legs, back and any combination thereof.

It is another object of the present invention to provide the system as defined above, wherein said system utilizes at least one selected from a group consisting of mechanical visualization, OCT, Ultrasound, machine learning algorithms, artificial intelligence, image processing and any combination thereof to efficiency select the preferred location of the tissue to be treated to enhance outcome of said treatment.

It is another object of the present invention to provide the system as defined above, wherein said stabilizing means are adapted to stabilize said at least one scar-like fibrous tissue generated.

It is another object of the present invention to provide the system as defined above, wherein said stabilizing means are adapted to perform application of at least one selected from a group consisting of RF energy, pulsed electromagnetic field, microwave, optical energy, ultrasound, and any combination thereof.

It is another object of the present invention to provide the system as defined above, additionally comprising means for application of at least one thread to stabilize said at least one scar-like fibrous tissue generated.

It is another object of the present invention to provide the system as defined above, additionally comprising at least one cutting element, integrated within said skin interference instrument, adapted to grind said excised tissue so as to facilitate extraction thereof.

It is another object of the present invention to provide the system as defined above, wherein said at least one skin interference instrument is in communication with at least one RF generator, adapted to apply RF energy to the skin and tissue, so as to fractional ablate/coagulate the tissue.

It is another object of the present invention to provide the system as defined above, wherein said application of RF energy is either simultaneously or sequentially with said at least one skin interference instrument.

It is another object of the present invention to provide the system as defined above, wherein said at least one skin interference instrument is in communication with at least one pulsed electromagnetic frequency generator.

It is another object of the present invention to provide the system as defined above, wherein said pulsed electromagnetic frequency generator is adapted to provide at least one dynamic magnetic field pulses to said skin.

It is another object of the present invention to provide the system as defined above, wherein said dynamic magnetic field pulses are provided by means of at least one coil.

It is another object of the present invention to provide the system as defined above, wherein said at least one skin interference instrument is at least partially coiled by at least one coil.

It is another object of the present invention to provide the system as defined above, wherein said at least one skin interference instrument is adapted to simultaneously provide both said electromagnetic pulses and said RF energy to said skin.

It is another object of the present invention to provide the system as defined above, wherein said RF energy results in heating said skin.

It is another object of the present invention to provide the system as defined above, wherein application of at least one energy selected from a group consisting of laser, pulsed electromagnetic field, RF, coblation, coagulation, ablation, microwave energy, ultrasound, application of any other type of energy and any combination thereof is either simultaneously or sequentially applied with said at least one skin interference instrument.

It is another object of the present invention to provide the system as defined above, wherein at least one of the following is being held true (a) the shape of said electromagnetic pulse is selected from the group consisting of square wave, a sine wave, a triangular wave, sawtooth wave, ramp waves, spiked wave or any combination thereof; (b) the magnetic field intensity B of each pulse applied by said pulsed electromagnetic frequency generator ranges between about 0 and about 3 Tesla; (c) the magnetic field intensity B of each pulse applied by said pulsed electromagnetic frequency generator ranges between about 0 to 40 Gauss; (d) the duration of each pulse applied by said pulsed electromagnetic frequency generator ranges between about 3 and about 1000 milliseconds; (e) the frequency F applied by the pulses of said pulsed electromagnetic frequency generator ranges between about 1 Hz and about 40 MHz; (f) the energy E applied by the pulses of said pulsed electromagnetic frequency generator ranges between about 1 and about 150 watts per pulse or any combination thereof; (g) the frequency F applied by the pulses applied is higher than about 1 and lower than about 1M Hz; (h) the frequency F applied by said electromagnetic field pulses ranges between 1 Hz and 50 Hz; (i) the frequency of said RF energy ranges between 200 kHz and 10 MHz; (j) the power P applied by said RF energy pulses ranges between 1 W and 100 W of RMS average power; and any combination thereof.

It is another object of the present invention to provide the system as defined above, additionally comprising at least one temperature sensor.

It is another object of the present invention to provide the system as defined above, additionally comprising a mechanism for skin cooling, adapted to regulate the temperature of the skin.

It is another object of the present invention to provide the system as defined above, wherein the distal end of said at least one skin interference instrument additionally comprising at least one selected from a group consisting of at least one impedance, at least one temperature sensor and any combination thereof.

It is another object of the present invention to provide the system as defined above, wherein said at least one selected from a group consisting of at least one impedance, at least one temperature sensor and any combination thereof is adapted to provide indication as to the depth of penetration of each of said at least one skin interference instrument.

It is another object of the present invention to provide the system as defined above, wherein said at least one skin interference instrument additionally comprising at least one needle, adapted to provide at least one treatment substance to the treatment area.

It is another object of the present invention to provide the system as defined above, additionally means adapted to at least partially severing at least one scar-like fibrous tissue.

It is another object of the present invention to provide the system as defined above, wherein said means for forming at least one interference of at least one tissue portion in said region of skin, are adapted to at least partially severing at least one scar-like fibrous tissue.

### DETAILED DESCRIPTION OF THE INVENTION

This invention relates to methods and devices for the treatment of the skin. Such treatment is provided by the generation of scar-like fibrous tissue (e.g., scar tissue, septae) by means of tissue interference (e.g., tissue incision and/or tissue excision, as defined below).

According to a preferred embodiment of the present invention, the device and method of the present invention discloses the deliberately generation of scar-like fibrous tissue (e.g., scar tissue) in predetermined locations and at a preterminal density. As, such scar-like fibrous tissue/scar tissue generated by the present invention results in having substantially the same properties as fibrous septae, such generation of scar tissue is in-fact generation of septae.

More specifically, the present invention relates to tissue engineering and the generation of at least one fibrous tissue (scar-tissue septa-like). Such generation of at least one fibrous tissue (scar-tissue septa-like) is provided by forming at least one interference of at least one tissue portion. Such interference is provided by tissue excision/incision and/or coring and/or any destruction or disruption to the tissue.

Tissue excision/incision and/or coring and/or any destruction or disruption to the tissue can be performed by fractional ablation of the epidermal and/or dermal layer of the skin with at least one hollow coring needle (or punch), by fractional laser ablation, by fractional radiofrequency (also refers to as RF), either by one or multiple RF electrodes, ablation, and/or by fractional ultrasonic ablation (using ultrasound), application of temperature to heat, application of laser, insertion of threads, RF, pulsed electromagnetic field, coblation, ablation, coagulation, application of heat to accelerate collagen synthesis in the tissue, microwave energy, any destruction or disruption to the tissue, application of any other type of energy and any other type of energy, any additives to the tissue and any combination thereof.

Thus, according to one embodiment, the device of the present invention creates interference of the tissue in a predetermined skin portions.

According to one embodiment, the device of the present invention excises patterns of dermal skin cores at desired density, and direction. Optionally, those holes are stabilized by, e.g., application of RF energy, PEMF (pulse electromagnetic pulses), ultrasound energy, microwave etc.

It is noted that stabilization refers hereinafter to the increase of the yield of collagen synthesis, yielding thicker/denser scar tissue.

As another alternative, the remaining holes in the skin are closed using manual compression methods such as compression tape, glue or tunable dressings.

According to one embodiment of the present invention, the device of the present invention is designed for the creation of skin micro-interference in a fractional manner.

According to one embodiment of the present invention, the device of the present invention is designed for the removal of skin micro-cores in a fractional manner.

### Definitions

The term “scar-like fibrous tissue” or “septae” refers hereinafter to bundles of dense connective tissue resembling dense scar, interconnecting the dermis, through the hypodermis, with the fascia layer or with hypodermal connective tissue. Fibrous septae are like narrow, semi-rigid bands that pull the skin downwards at anchoring points.

The main role of the connective tissue of the dermis is to provide mechanical strength and elasticity so to maintain structure, isolation and stability of the subcutaneous and deeper zones. The connective tissue is mainly composed of:
collagen which gives tensile strength; and,
elastic tissue that gives the skin the ability to stretch and return to normal.

The term “scar tissue” refers hereinafter to dense fibrous tissue that is being formed when normal tissue is affected beyond threshold or destroyed by endogenic or exogenic impacts or interventions such as disease, injury, physical or chemical intervention or surgery. For example, scar tissue is formed as part of the common wound healing process when a wound is formed by a cut, sore, burn, or other skin condition, or when an incision (cut) is made into the skin during surgery. It may also be formed inside the body when certain conditions, such as cirrhosis, cause normal tissue to become fibrotic tissue.

The term “about” refers hereinafter to +/−25% of any recited value.

The term “overlap” refers hereinafter to vertex, facet, cross sectional area and any combination thereof.

The term “Optical coherence tomography (OCT)” refers hereinafter to a non-invasive imaging. In other words, OCT is an imaging technique that uses low-coherence light to capture micrometer-resolution, two- and three-dimensional images from within optical scattering media (e.g., biological tissue). It is used for medical imaging and industrial nondestructive testing (NDT). Optical coherence tomography is based on low-coherence interferometry, typically employing near-infrared light. The use of relatively long wavelength light allows it to penetrate into the scattering medium. Confocal microscopy, another optical technique, typically penetrates less deeply into the sample but with higher resolution.

The term “mechanical visualization” refers hereinafter to either the use of ultrasound or OCT to image the under surface of the treated area skin/tissue. Such mechanical visualization is used to efficiency select the preferred location of the tissue to be treated to enhance outcome of said treatment. It should be noted that according to the present invention the term ‘mechanical visualization’ also includes 2D and 3D cameras for imaging the surface of the treated area skin/tissue.

The term “skin” refers hereinafter to the largest organ of the body. The skin protects us from microbes and the elements, helps regulate body temperature, and permits the sensations of touch, heat, and cold. The skin has several layers:
The epidermis, the outermost layer of skin.
The dermis, beneath the epidermis, contains tough connective tissue, hair follicles, and sweat glands.
The deeper subcutaneous tissue (hypodermis) is made of fat and connective tissue and is up to the fascia.

According to the present invention the term “skin” refers to all 3 layers (up to the fascia) or any portion thereof.

The term “interference” refers hereinafter to either incision/incised tissue and/or excision/excised tissue and/or coring (by means selected from of mechanical means, blades, one or more solid needles, application of temperature to heat and evacuate tissue, application of temperature to heat, application of laser, insertion of threads, pulsed electromagnetic field, RF, coblation, coagulation, ablation, microwave energy, application of heat to accelerate collagen synthesis in the tissue, ultrasound, fractional laser ablation, fractional radiofrequency ablation, coblation, coagulation, microwave energy and/or fractional ultrasonic ablation, application of any other type of energy and any combination thereof).

The term “incised” tissue portion or “incision” refers hereinafter to any destruction or disruption to the tissue such as a cut, abrasion, ablation or coagulation of tissue, including a tissue portion in a skin region, or the act of cutting, abrading, or ablating tissue, in a skin region, or one or more tissue portions. For example, an incision includes any cut, abrasion, or ablation into tissue, which can result in destruction of tissue or a portion thereof and, thereby, produce one or more holes or slits in the skin region. Exemplary methods of forming incised tissue portions or incisions include use of one or more blades, one or more solid needles, fractional laser ablation, fractional radiofrequency ablation, coblation, coagulation, microwave energy and/or fractional ultrasonic ablation, any useful tool for forming tissue destruction or incisions, or any methods and apparatuses described herein.

The term “excised” tissue portion or “excision” refers hereinafter to a removed tissue, including a tissue portion from a skin region, or the act of removing tissue or one or more tissue portions from a skin region. Excision is usually referred to as “to surgically remove”. This term is often used in reference to removing a mass, excision means that tissue is removed, using a scalpel, laser, coblation, coagulation, ablation, ultrasound, microwave energy, RF, application of heat (to evaporate skin portions), mechanical applicator that ‘drills’ (cores) through the skin whilst suction is applies (during the drilling/coring or thereafter) to remove the to be excised skin portion, or any other instrument. For example, an excision includes any removed tissue or tissue portion from a skin region, which can result in excised tissue portions having a particular geometry (e.g., a cylindrical geometry, rectangular, triangle etc. or any arbitrary shape) and produce one or more holes (i.e., negative space created by the removal of tissue) in the skin region. Exemplary methods of forming excised tissue portions or excisions include use of one or more hollow needles (optionally include one or more notches, extensions, protrusions, and/or barbs), one or more microaugers, one or more microabraders, vacuum, any ablative means (including ablative lasers etc.)—may be used for incision and for excision, any useful tool for forming excisions, or any methods and apparatuses described herein.

The term “application of compression forces” refers hereinafter to a physical change in the compression tape (as disclosed hereafter). In this case, the forces applied are compression forces to compress a tape (e.g., TegaDerm®).

The term “application of expansion forces” refers hereinafter to a physical change in the compression tape (as disclosed hereafter). In this case, the forces applied are stretching forces to expand the tape.

It is emphasized that the following disclosure provides an example of interference instrument being coring means (e.g., punches/needles), however, any other example that results in either incision/incised tissue and/or excision/excised tissue and/or coring (by means selected from of mechanical means, blades, one or more solid needles, application of temperature to heat and evacuate tissue, application of temperature to heat, application of laser, insertion of threads, application of heat to accelerate collagen synthesis in the tissue, pulsed electromagnetic field, RF, coblation, coagulation, ablation, microwave energy, ultrasound, fractional laser ablation, fractional radiofrequency ablation, coblation, coagulation, microwave energy and/or fractional ultrasonic ablation, application of any other type of energy and any combination thereof) is within the scope of the present invention.

According to one embodiment of the present invention, the interference mechanism is a cartridge consisting of at least one, up to 0.75 mm in diameter, hollow needles (or punches) inserted into the skin while rotating at a range of 2000-10000 RPM with a maximum penetration depth of up to 10 mm.

According to one embodiment of the present invention, the interference mechanism is the applications of additives (e.g., threads) inserted into the treated tissue. Thus, according to this embodiment the interference to the skin is performed by insertion (and securing) threads into the skin (up to the fascia tissue) in a predetermined pattern (e.g., amount, density, orientation etc.).

As described above, this invention further relates to methods and devices for skin treatment. More, specifically, this invention relates to methods and devices for skin interference (e.g., coring, incision/incised tissue and/or excision/excised tissue) that would promote collagen growth in the generation of fibrous tissue (scar-tissue septa-like).

Thus, the device could be utilized in a wide variety of fields e.g., skin laxity, skin resurfacing, cheek wrinkles treatments, wrinkles treatments, folds treatments, acne scars removal, dyschromia treatment, striae treatment, surgical or burn scars removal, tattoos removal and any combination thereof.

According to one embodiment, treatment area is selected from a group consisting of forehead, cheeks, jaw line, neck, upper arms, tummy, abdomen, face, eyelid, nose, forehead, chin, forehead, lips, nose, thighs, chest, legs, back and any combination thereof.

In particular embodiments, the present invention provides one or more of the following advantages. First, the methods and devices herein enable visualization of results in real time during the course of the treatment. One can envision asking the patient for feedback in real time during the treatment and adjusting the tightening to the patient preference. Second, the methods and devices herein require less skill than that of a surgeon. One can envision treatment of patients in an outpatient setting, rather than requiring an inpatient, surgical setting. Third, the methods and devices herein constitute minimally invasive techniques, which can provide more predictable results and/or lower risk factors than that for more invasive techniques (e.g., plastic surgery) or non-invasive energy-based techniques (e.g., laser, coblation, ablation, coagulation, microwave energy, radiofrequency, or ultrasound). Finally, the methods and devices herein can be useful for maximizing the tightening effect, while minimizing healing time by optimizing tightening (e.g., by controlling the extent of skin pleating, such as by increasing the extent of skin pleating for some applications or skin regions and by decreasing the extent of skin pleating for other applications or skin regions, as described herein).

The device of the present invention is designed to treat the tissue by tissue engineering, as well as to enhance quality and productivity of skin laxity reduction procedures using advanced robotics, including tissue interference (incision/excision/coring means), machine vision and advanced software.

The device implements skin interference instrument adapted to incise tissue and/or excise tissue and/or core the tissue approach to create new dense fibrous tissue (scar-tissue septa-like). The device excises a pattern of predetermined size of dermal septae like scar tissue at desired depth, density, and direction. The performed remaining destructed tissue (and/or holes) in the skin are then stabilized (by wound healing process leading to demarcate the septae-like scar tissue. Such wound healing include hemostasis, inflammation, collagen synthesis and maturation processes) to substantially function as septae like scar tissue that stabilizes or tightened the skin.

It is noted that stabilization refers hereinafter to the increase of the yield of collagen synthesis, yielding thicker/denser scar tissue.

Optionally the destructed tissue (and/or holes) are closed using manual compression methods such as compression tape or glue.

According to one embodiment of the present invention, the treatment parameters; i.e., desired density of the interfered tissue, excised tissue, incised tissue, cores, depth, diameter, angle of said at least one robotic arm, orientation of new scar which will act as new septae like scar tissue etc. are automatically adjusted to the treated patient. Alternatively, the treatment parameters are manually inserted into the device specifically and tailor-made to the treated patient.

According to one embodiment of the present invention, the interference mechanism is the applications of additives (e.g., threads) inserted into the treated tissue. Thus, according to this embodiment the interference to the skin is performed by insertion (and securing) threads into the skin in a predetermined pattern (e.g., amount, density, orientation etc.).

According to another embodiment, the device may include a stretching/compression device (e.g., compression tape) that will enable stretching/compression of the skin, post the interference stage.

Various modifications and variations of the described method and system of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, pharmacology, or related fields are intended to be within the scope of the invention.

1.-135. (canceled)

136. A method for treating a patient's skin, comprising:
i. identifying at least one region of said patient's skin in need with treatment; and,
ii. forming at least one interference of at least one tissue portion in said region of skin, thereby generating at least one scar-like fibrous tissue in said region of said patient's skin;
wherein said step of generating at least one scar-like fibrous tissue in said region of said patient's skin treats said at least one region.

137. The method of claim 136, wherein said step of forming at least one interference of at least one tissue portion results in generating a plurality of scar-like fibrous tissue in said region of skin, each at a different angle, relative to each other and said skin.

138. The method of claim 136, wherein said step of forming at least one interference of at least one tissue portion results in the generation of at least one crisscross structure in said at least one tissue portion.

139. The method of claim 136, additionally comprising step of forming at least one interference of at least one tissue portion in said region of skin, wherein said step of forming at least one interference of at least one tissue portion in said region of skin is performed by a system comprising at least one robotic arm, said at least one robotic arm comprising at least one skin interference instrument selected from a group consisting of at least one needle, at least one punch, at least one thread, and any combination thereof, and said at least one skin interference instrument is configured to contact a surface of the skin to generate at least one interference in the skin tissue.

140. The method of claim 139, further comprising adjusting translation or rotation of said at least one robotic arm relative to said skin as said at least one robotic arm approaches said skin, wherein said angle of said at least one robotic arm is in the range of about 0 to about 90 degrees.

141. The method of claim 139, wherein each one skin interference instrument rotates individually in a predefined direction in a predetermined speed, at least two of said at least one skin interference instrument rotate simultaneously, each one skin interference instrument translates individually, at least two of said skin interference instruments translate simultaneously, or a distance between each pair of neighboring skin interference instruments is configured to vary and be adjustable either before or during treatment.

142. The method of claim 136, additionally comprising sensing contact with said skin or cooling the skin.

143. The method of claim 139, further comprising utilizing a controller to control the at least one robotic arm, wherein the controller is adapted to define at least one no-fly zone; said no-fly zone being defined as an area to which said system provides no treatment.

144. The method of claim 136, additionally comprising delivering additives to the skin, wherein said additives are selected from a group consisting of threads, therapeutic agents, anesthesia, saline solution growth factors, platelet-derived growth factor (PDGF), transforming growth factor beta (TGF-β), fibroblast growth factor (FGF), epidermal growth factor (EGF), and keratinocyte growth factor); one or more stem cells; steroids, agents which prevent post-inflammatory skin hyperpigmentation, hydroquinone, azelaic acid, kojic acid, mandelic acid, or niacinamide; one or more analgesics; one or more antifungals; one or more anti-inflammatory agents, or a mineralocorticoid agent, an immune selective anti-inflammatory derivative; one or more antimicrobials; a foam; or a hydrogel, one or more antiseptics, one or more antiproliferative agents, one or more emollients; one or more hemostatic agents, a procoagulant, an anti-fibrinolytic agent, one or more procoagulative, one or more anticoagulative agents, one or more immune modulators, including corticosteroids and non-steroidal immune modulators, one or more proteins; or one or more vitamins, hyaluronic acid, collagen, low melting agarose (LMA), polylactic acid (PLA), and/or hyaluronic acid, hyaluranon); a photosensitizer (e.g., Rose Bengal, riboflavin-5-phosphate (R-5-P), methylene blue (MB), N-hydroxypyridine-2-(1H)-thione (N-HTP), a porphyrin, or a chlorin, as well as precursors thereof); a photochemical agent, 1,8 naphthalimide); a synthetic glue (e.g., a cyanoacrylate adhesive, a polyethylene glycol adhesive, or a gelatin-resorcinol-formaldehyde adhesive), a biologic sealant and any combination thereof.

145. The method of claim 139, wherein said system additionally comprising at least one subsystem selected from a group consisting of (a) a vacuum subsystem adapted to apply suction to remove excising portions of said skin tissue; (b) at least one retainer, in communication with at least one excisor configured to produce a plurality of excised tissue portions, adapted to contain said excised tissue, to avoid the use of vacuum; (c) any combination thereof.

146. The method of claim 136, wherein said skin is part of a treatment area selected from a group consisting of buttocks, lower limbs, abdomen, forehead, cheeks, jaw line, upper arms, tummy, abdomen, face, eyelid, forehead, chin, forehead, lips, nose, neck, thighs, chest, legs, back and any combination thereof; furthermore, said treatment could be selected from a group consisting of skin laxity, skin resurfacing, cheek wrinkles treatments, wrinkles treatments, folds treatments, acne scars removal, dyschromia treatment, striae treatment, surgical or burn scars removal, tattoos removal and any combination thereof.

147. The method of claim 136, additionally comprising stabilizing said at least one scar-like fibrous tissue generated, wherein said step of stabilizing is performed by application of at least one selected from a group consisting of RF energy, a pulsed electromagnetic field to provide at least one electromagnetic pulse from a pulsed electromagnetic frequency generator, microwave, optical energy, cryo freezing, cryogenics, ultrasound, and any combination thereof.

148. The method of claim 147, wherein at least one of the following is satisfied: (a) the shape of said at least one electromagnetic pulse is selected from the group consisting of square wave, a sine wave, a triangular wave, sawtooth wave, ramp waves, spiked wave or any combination thereof; (b) the magnetic field intensity B of each pulse applied by said pulsed electromagnetic frequency generator ranges between about 0 and about 3 Tesla; (c) the magnetic field intensity B of each pulse applied by said pulsed electromagnetic frequency generator ranges between about 0 to 40 Gauss; (d) the duration of each pulse applied by said pulsed electromagnetic frequency generator ranges between about 3 and about 1000 milliseconds; (e) the frequency F applied by the pulses of said pulsed electromagnetic frequency generator ranges between about 1 Hz and about 40 MHz; (f) the energy E applied by the pulses of said pulsed electromagnetic frequency generator ranges between about 1 and about 150 watts per pulse or any combination thereof; (g) the frequency F applied by the pulses applied by said step of applying pulsed electromagnetic therapy to said region to be higher than about 1 and lower than about 1M Hz; (h) the frequency F applied by said electromagnetic field pulses ranges between 1 Hz and 50 Hz; (i) the frequency of said RF energy ranges between 200 kHz and 10 MHz; (j) the power P applied by said RF energy pulses ranges between 1 W and 100 W of RMS average power; and any combination thereof.

149. The method of claim 136, additionally comprising at least partially severing at least one scar-like fibrous tissue, wherein said step of at least partially severing at least one scar-like fibrous tissue is performed by said step of forming at least one interference of at least one tissue portion in said region of skin.

150. A system of treating a patient's skin, comprising:
(i) means for identifying at least one region of said patient's skin in need with treatment; and,
(ii) means for forming at least one interference of at least one tissue portion of skin thereby generating at least one scar-like fibrous tissue in said region of said patient's skin;
wherein said at least one scar-like fibrous tissue generated in said region of said patient's skin treats said at least one region.

151. The system of claim 150, wherein said means of forming at least one interference of at least one tissue portion comprising means for generating at least one scar tissue in at least one tissue portion in said region of skin, wherein said means of forming at least one interference of at least one tissue portion comprising at least one selected from a group consisting of means of excising at least one tissue portion; means of coring at least one tissue portion; means of incision of at least one tissue portion and any combination thereof.

152. The system of claim 150, wherein said means of forming at least one interference of at least one tissue portion results in generating a plurality of scar-like fibrous tissue in said region of skin, each at a different angle, relative to each other and said skin.

153. The system of claim 150, wherein said means of forming at least one interference of at least one tissue portion in said region of skin comprises means selected from a group consisting of mechanical means, application of temperature to heat and evacuate tissue, application of temperature to heat, application of laser, RF, insertion of threads, pulsed electromagnetic field, application of heat to accelerate collagen synthesis in the tissue, coblation, ablation, coagulation, microwave energy, ultrasound, cryo freezing, cryogenics, any destruction or disruption to the tissue, application of any other type of energy, any additives to the tissue and any combination thereof.

154. The system of claim 150, wherein said means of forming at least one interference of at least one tissue portion in said region of skin comprising a system comprising at least one robotic arm, said at least one robotic arm comprising at least one skin interference instrument comprising a plurality of punches configured to contact a surface of the skin to generate holes in the skin tissue.

155. The system of claim 154, further comprising a stopper adapted to limit the depth to which said at least one skin interference instrument penetrates said skin.

156. The system of claim 154, additionally comprising at least one sensor adapted to indicate contact with said skin.

157. The system of claim 154, wherein said skin is a treatment area selected from a group consisting of the buttocks, lower limbs, abdomen, forehead, cheeks, jaw line, neck, upper arms, tummy, abdomen, face, eyelid, nose, forehead, chin, forehead, lips, nose, neck, thighs, chest, legs, back and any combination thereof; furthermore, said treatment could be selected from a group consisting of skin laxity, skin resurfacing, cheek wrinkles treatments, wrinkles treatments, folds treatments, acne scars removal, dyschromia treatment, striae treatment, surgical or burn scars removal, tattoos removal and any combination thereof.

158. The system of claim 154, additionally comprising at least one cutting element, integrated within said skin interference instrument, adapted to grind said excised tissue so as to facilitate extraction thereof.

159. The system of claim 154, wherein application of at least one energy selected from a group consisting of laser, pulsed electromagnetic field, RF, coblation, coagulation, ablation, microwave energy, ultrasound, application of any other type of energy and any combination thereof is either simultaneously or sequentially applied with said at least one skin interference instrument.

160. The system of claim 154, wherein the distal end of said at least one skin interference instrument additionally comprises at least one impedance element or at least one temperature sensor, which is configured so as to indicate the depth of penetration of said at least one skin interference instrument.
